(19) United States
(12) Patent Application Publication
(10) Pub. No.: US 2024/0148293 A1
(43) Pub. Date: May 9, 2024

(54) SAMPLING AND/OR INJECTING DEVICE

(71) Applicant: Human Holding ApS, København N (DK)

(72) Inventors: Andreas Christian TUXEN, København N (DK); Victor Leth STAALKJAER, København N (DK)

(73) Assignee: Human Holding ApS, København N (DK)

(21) Appl. No.: 17/981,538

(22) Filed: Nov. 7, 2022

(51) Int. Cl. A61B 5/15

(52) U.S. Cl. CPC A61B 5/150022; A61B 5/150412; A61B 5/15107

(57) ABSTRACT

A sampling and/or injecting device (100) comprising an elongated tubular housing (101) and a sampling and/or injecting arrangement (105) comprising a piercing element (106) configured to engage a sampling and/or injecting area (103) and a sampling and/or injecting element (107) configured for automatically collecting a body fluid sample from said sampling and/or injecting area (103) and/or injecting a substance in said sampling and/or injecting area (103). A securing element (102) is disposed at a distal end of the tubular housing (101) and a sealing element (110) is disposed within or around the securing element (102). An activator (113) is configured to activate automatic movement of the piercing element (106) to and/or from the sampling and/or injecting area (103) after attachment of the securing element (102) to the sampling and/or injecting area (103). A mechanical resistor (114), controlling the automatic movement of the piercing element (106), is mechanically or pneumatically operably connected to the activator (113).

### TECHNICAL FIELD

The disclosure relates to a device for collecting a body fluid sample and/or for injecting a compound into a body, particularly to a device and method for fetal and/or neonatal blood sampling during labor, e.g. for determining oxygen saturation of fetal and/or neonatal blood.

### BACKGROUND

Each year 130 million births take place globally. According to the World Health Organization, between 4 and 10 million newborns develop birth asphyxia each year. Of these, approximately 1.2 million die and about the same number of newborns suffer from severe brain damage and brain death due to oxygen deprivation in the fetus or neonate during labor. This may have severe consequences, such as epilepsy, cerebral palsy, and developmental delays.

Fetal monitoring refers to a technology used for monitoring the well-being of a fetus during labor. Such monitoring may serve a preventive purpose by providing a basis for deciding how to best proceed during labor. For example, whether a caesarean section (C-section) should be performed, whether vacuum extraction for assisting the delivery should be performed, or whether other means of securing the fetus' well-being and facilitating delivery are needed. Performing a C-section is a surgical intervention which should be avoided if possible, for both health reasons and economic reasons. Proper monitoring of the fetus' state may help to reduce the number of unnecessary C-sections. Further, fetal monitoring may serve a documentation purpose by generating health data about fetuses and newborns, which data can be fed into national healthcare systems, e.g. for statistical health and clinical performance analysis.

Different technologies for fetal monitoring exist, including fetal scalp blood sampling which is a technique by which capillary blood is sampled from the fetal scalp during labor for determining whether fetal oxygenation is sufficient. Commonly, the pH and/or the lactate in the sampled blood is measured. A low pH and high level of lactate indicate acidosis which is associated with hypoxia (i.e. deprivation of adequate oxygen supply). Fetal scalp blood sampling is generally performed by creating a shallow cut in or puncturing the fetal scalp using a transvaginally inserted blood lancet, followed by applying a thin pipe/tube or capillary tube to the incision site for drawing a blood sample.

The current practice for fetal scalp blood sampling is often rather cumbersome and time-consuming for healthcare personnel performing the procedure (typically midwives, nurses, or doctors who have to attend to multiple patients within a single day) and uncomfortable for the patient (the woman in labor). Most known techniques require visual access to the fetus' scalp. Generally, a cone-shaped amnioscope with a light source is used for viewing the fetus' scalp therethrough. The amnioscope is inserted into the vagina, with the narrow end of the amnioscope resting against the presenting part of the scalp of the fetus, and the lancet and tube are manually passed through the amnioscope for making an incision in the fetal scalp and drawing a blood sample. However, proper and secure attachment of the amnioscope to the scalp is often challenging, e.g. due to fetal movement.

One instrument for obtaining a fetal blood sample is shown in EP 0166574 A2. The instrument has an elongated member having an open end portion and a membrane disposed over the open end portion (14) for perfecting a seal about the open end portion. A lancet in the form of a blade or a sharp ended capillary tube is disposed in the elongated member for piercing the membrane and making an incision in the skin of a fetus. A plunger is supported within the elongated member and is movable to create a suction within the open end portion to draw fetal blood from the incision through the pierced membrane (and into the open end portion.

Contamination of the blood to be sampled is undesirable. Therefore, known procedures for fetal scalp blood sampling generally include steps of manually cleaning the incision and sampling site from debris and blood in the surrounding environment prior to incision and sampling. Further, the incision and sampling site needs to be kept clean during the incision and sampling phase. It is important that only fetal blood is sampled and to avoid air contamination of the sampled blood, since the presence of air bubbles in the blood represents a source of analytical error in pH and lactate measurement and, thus, may lead to flawed conclusions. Another complication of many current practices for fetal scalp blood sampling is that the sampling equipment used is often too big for application in the early birth stage.

There is thus a need in the art for an improved device and method for fetal scalp blood sampling.

### SUMMARY

It is an object provide an apparatus for fetal scalp blood sampling to overcome or at least reduce the problems mentioned above.

The foregoing and other objects are achieved by the features of the independent claims. Further implementation forms are apparent from the dependent claims, the description, and the figures.

According to a first aspect, there is provided a sampling and/or injecting device comprising an elongated tubular housing, which tubular housing is at least partly bendable, and a sampling and/or injecting arrangement configured to engage a sampling and/or injecting area, the sampling and/or injecting arrangement comprising a piercing element configured to engage the sampling and/or injecting area, and a sampling and/or injecting element configured for automatically collecting a body fluid sample from the sampling and/or injecting area and/or configured for injecting a substance in the sampling and/or injecting area, characterised in that the sampling and/or injecting device further comprises a securing element disposed at a distal end of the tubular housing, which securing element is operably connected to a lumen of the tubular housing and configured to attach with an open end to a sampling and/or injecting area, a sealing element disposed within or around the securing element and configured to seal the sampling area, an activator configured to activate automatic movement of the piercing element to and from the sampling and/or injecting area, after attachment of the securing element (to the sampling and/or injecting area, and a mechanical resistor for controlling the automatic movement of the piercing element, the mechanical resistor being mechanically or pneumatically operably connected to the activator.

Such a device allows a sample to be taken with a reduced, if not completely eliminated, risk of contamination of the sampling and/or injecting area. Furthermore, the device allows the procedure to be far more comfortable for the patient, in particular in cases where the device is inserted into a body orifice. Additionally, the device is easy and quick to use, and provides very secure attachment to the sampling and/or injecting area.

In a possible implementation form of the first aspect, activator is disposed at a proximal end of the tubular housing.

In a further possible implementation form of the first aspect, the device is activated mechanically or pneumatically.

In a further possible implementation form of the first aspect, the securing element and the elongated tubular housing are configured to be at least partly inserted into a body orifice.

By providing an elongated tubular housing, a securing element, and a sealing element that is insertable into a body orifice such as the birth canal, it becomes possible to take fetal blood samples in a safe, easy, quick, and secure manner. The device does not only provide a more comfortable user experience both for the expectant mother in labor and their healthcare professionals, but the risk of contamination is significantly reduced, if not completely eliminated due to the sealing element. By providing an activator and a mechanical resistor it becomes possible to precisely control the sampling/injecting arrangement at an environment, where there is usually little space and where fetal movement may make it difficult to collect samples or inject medicaments during labor.

In a further possible implementation form of the first aspect, the device is configured for sampling a body fluid from a fetus and/or neonate, and/or for injecting a compound into a fetus and/or neonate, and the sampling and/or injecting area is one of a scalp area and a heel area of the fetus or neonate.

In a further possible implementation form of the first aspect, the piercing element is a lancet or a needle for making an incision in and/or for puncturing the sampling and/or injecting area.

By incorporating the piercing element to the device, it becomes possible to significantly advance on the current means and methods for pH/lactate detection during labor and to allow the safe use of a single device, as all current methods require the use of several equipment (e.g. an aminoscope, swabs, gel, cutting knife etc.) to take a contamination-free sample. While current applications require the use of several tools for every function (e.g. the knife component is only used to enter the skin and is then disposed), the separation of processes increases the necessary steps and hence the length of the sampling procedure, potentially influencing the sample quality and usability for analysis. Additionally, every time the user switches between the tools (e.g. from the cutting knife to a swab or capillary tube), the new tool has to be steered back into the sampling area, increasing the chances of contamination and mistakes. By providing a sampling tool that incorporates all required tools (e.g. a piercing element, a sealing element, a sampling and/or injecting arrangement etc.) in a single hand-held equipment, it becomes possible to take a sample quickly and precisely even when the fetus is not yet visible in the birth canal.

In a further possible implementation form of the first aspect, the sampling and/or injecting element is a tube, preferably a capillary tube, or a swab configured for collecting a body fluid sample from the sampling and/or injecting area, and/or a tube configured for injecting a substance in the sampling and/or injecting area.

In a further possible implementation form of the first aspect, the sampling and/or injecting element is provided at least partly within the tubular housing.

By providing a sampling and/or injecting element within the sampling and/or injecting arrangement it becomes possible to very precisely collect the required sample from the sampling and/or injecting area. This not only reduces the time needed for collecting a sample in an anyways rather stressful and inconvenient environment, such as during labor, but also allows for causing less pain and distress to both the fetus and the mother in labor.

In a further possible implementation form of the first aspect, a capillary effect is created in the sampling and/or injecting element, and the body fluid is drawn from the sampling and/or injecting area into the sampling and/or injecting element by the capillary effect.

By utilizing a capillary effect to draw up the required body fluid, the required sample of body fluid can be collected quickly and precisely from the sampling and/or injecting area.

In a further possible implementation form of the first aspect, the securing element comprises a substantially cup-shaped outer and/or inner surface.

The securing element may have various shapes, forms and configurations, suitable for safely mounting the device onto the sampling and/or injection area. The securing element may further comprise an inner and/or outer surface for attaching the device securely onto e.g. the scalp of the fetus during labor.

In a further possible implementation form of the first aspect, at least part of the securing element is made of a flexible material.

In a further possible implementation form of the first aspect, the tubular housing and the securing element are made substantially of plastic.

By implementing the use of flexible and/or plastic materials, e.g. medical grade plastics, PVC, polypropylene, polyethylene, polycarbonate, polysulfone etc. or the formulation of custom polymers has the advantage of being bendable and hence the tubular housing containing the securing element fits into the birth canal during sampling of a fetus during labor more easily, causing less inconvenience to the mother in labor. Due to the natural curvature of the birth canal and the entrance into the cervix, the currently employed amnioscopes are too large for use in the early phase of birth, as it requires 3-4 cm dilation of the cervix. This causes a problem during the early stages or birth or during more complicated births. Furthermore, the lack of available testing of fetal Lactate and/or oxygen levels during labor often results in having to perform a C-section.

In a further possible implementation form of the first aspect, the piercing element and the sampling and/or injecting element each extend along a longitudinal axis of the tubular housing.

In a further possible implementation form of the first aspect, the sampling and/or injecting element is configured for guiding the piercing element.

In a further possible implementation form of the first aspect, the sealing element has an aperture for allowing the piercing element and/or the sampling and/or injecting element to pass through to the sampling and/or injecting area.

By guiding the piercing element with the sampling and/or injecting element it becomes possible to precisely puncture the sampling and/or injecting area for creating a blood droplet that can be collected for analysis, even if the e.g. scalp of the fetus is not yet visible during the early stages of birth.

In a further possible implementation form of the first aspect, at least part of the device is disposable.

In a further possible implementation form of the first aspect, the device is substantially symmetrical along its longitudinal axis.

In a further possible implementation form of the first aspect, the piercing element and/or the sampling and/or injecting element are supported in a spring-loaded manner by the activator and/or the mechanical resistor.

The activator and/or mechanical resistor allows for a precise sampling and/or injecting as the user can ensure that upon identification of the required sampling and/or injecting area, the activator and/or mechanical resistor activates the piercing element for puncturing the skin and/or the sampling and/or injecting element in a controlled, linear movement for collecting the body fluid. Hence, it becomes possible for the previously segmented, multi-step procedure to be performed quicker and with less risk of making a sampling error.

In a further possible implementation form of the first aspect, the piercing element and the sampling and/or injecting element are operably coupled to one-another.

In a possible implementation form of the first aspect, the device further comprises a handle disposed at a proximal end of the tubular housing for holding the device.

In a possible implementation form of the first aspect, the securing element is configured to attach to the sampling and/or injecting area by means of friction forces between the securing element and the sampling and/or injecting area.

In a further possible implementation form of the first aspect, the device further comprises a vacuum generating arrangement for generating a vacuum within the securing element, the vacuum generating arrangement optionally being activated by the activator.

In a further possible implementation form of the first aspect, the vacuum generating arrangement is configured to attach the securing element to the sampling and/or injecting area, and, optionally, to increase a flow of body fluid to the sampling and/or injecting area by vacuum forces.

In a further possible implementation form of the first aspect, the vacuum generating arrangement is configured such that the vacuum is generated in a first step, attaching the securing element to the sampling and/or injecting area, and in a second step, after the sampling and/or injecting arrangement engaging the sampling and/or injecting area, increasing the flow of body fluid to the sampling and/or injecting area by means of the vacuum, the second step immediately following the first step.

By utilizing friction or vacuum forces it can be ensured that the securing element safely attaches to the sampling and/or injecting area. This has several advantages: not only is the sampling and/or injecting area sealed off from the external environment by preventing liquid inflow and allowing for a contamination-free sampling, but it is also ensured that in an otherwise confined space, which is often not yet visually visible and may be influenced by movement of the fetus and/or the mother, the sampling can be done quickly and accurately, without displacing the device from the sampling and/or injecting area. Moreover, the utilization of vacuum draws the blood to the sampling area provides a non-invasive mounting to the sampling and/or injecting area.

In a further possible implementation form of the first aspect, the vacuum generating arrangement can be connected and disconnected from the securing element.

In a further possible implementation form of the first aspect, the handle comprises the activator and the vacuum generating arrangement.

In a further possible implementation form of the first aspect, the vacuum is applied to all of the sampling and/or injecting area enclosed by the securing element.

By providing a device for collecting a fetal and/or neonatal body fluid sample during labor, it becomes possible to quickly and accurately collect samples, having the potential to reduce the number of unnecessary C-sections currently performed due to the lack of available testing of lactate and/or oxygen levels during birth. The time required for sampling is significantly reduced and the precision of the sampling device and method allows for a quick, easy and convenient sampling even if the fetus is not yet visible in the birth canal.

### BRIEF DESCRIPTION OF THE DRAWINGS

In the following detailed portion of the present disclosure, the aspects, embodiments, and implementations will be explained in more detail with reference to the example embodiments shown in the drawings, in which:

FIG. 1 is a diagrammatic illustration of a sampling and/or injecting device in accordance with an embodiment of the present invention;

FIGS. 2a to 2i are diagrammatic illustrations of sampling and/or injecting devices, with and without a vacuum generating arrangement, in accordance with various embodiments of the present invention;

FIGS. 3a and 3b are diagrammatic illustrations the activator and mechanical resistor, and the possible configurations for the position of the piercing element and the sampling and/or injecting element, in accordance with possible embodiments of the present invention;

FIGS. 3a to 3b are diagrammatic illustrations of a sampling and/or injecting arrangement of an embodiment of a sampling and/or injecting device;

FIGS. 4a to 4c are diagrammatic illustrations of a sampling and/or injecting arrangement and the sampling and/or injecting element in accordance with an embodiment of the present invention;

FIGS. 5a to 5d are diagrammatic illustrations of a method for collecting a fetal and/or neonatal body fluid sample during labor using a sampling and/or injecting device in accordance with an embodiment of the present invention;

FIGS. 6a to 6h are diagrammatic illustrations of a method for fetal scalp blood sampling during labor using a sampling and/or injecting device in accordance with an embodiment of the present invention;

FIG. 7 is a flowchart of a method for collecting a body fluid sample using a sampling and/or injecting device in accordance with an embodiment of the present invention.

### DETAILED DESCRIPTION

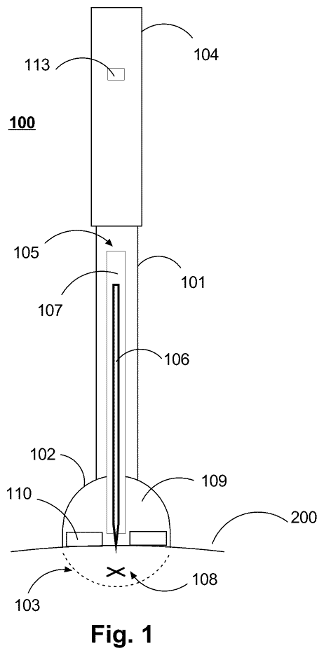

FIG. 1 shows a sampling and/or injecting device 100 in accordance with an embodiment. The device comprises an elongated tubular housing 101, which tubular housing 101 is at least partly bendable, and a securing element 102 disposed at a distal end of the tubular housing 101, with the securing element 102 operably connected to a lumen of the tubular housing 101. The securing element 102 attaches with its open end to a sampling and/or injecting area 103.

The securing element 102 is configured to attach to the sampling and/or injecting area 103 by friction forces between the securing element 102 and the sampling and/or injecting area 103. Thus, relative motion of the securing element 102 with respect to the sampling and/or injecting area or surface 103 is diminished, and the securing element 102 maintains a secured attachment position even during movement of the fetus and/or the mother.

The securing element 102 can also be configured to attach to the sampling and/or injecting area 103 by vacuum forces created within the securing element and the surface of the skin 200. Hence, the securing element 102 functions as a suction cup or sucker, using negative fluid pressure for adhering to the sampling and/or incision area 103. Particularly, when the securing element 102 is mounted on the sampling and/or injecting area 103, the volume of the space 109 between the securing element 102 and the skin surface 200 decreases, causing fluids such as air, water, mucus, etc. present between the securing element 102 and sampling and/or injecting area 103 to be expelled of the securing element 102, creating partial or full vacuum enclosed within the securing element 102 and the skin surface 200.

The securing element 102 is made substantially of elastic, flexible material such as rubber or plastic, and several suitable shapes are identified in the drawings. The securing element 102 may, as shown in FIG. 1, have a substantially cup-shaped outer and inner surface.

A sealing element 110 is disposed within or around the securing element 102. The sealing element 110 ensures that the sampling and/or injecting area 103 is sealed off of the rest of the environment to ensure that the required sample is not contaminated. The sealing element 110 may be made of rubber, plastic, or any other suitable material for securely sealing off the cavity and/or space 109 between the securing element 102 and the surface of the skin 200 to create partial or full vacuum or to adhere to the surface of the skin 200 through friction forces. The sealing element 110 can also be configured to remove liquids present in the sampling and/or injecting area 103 by e.g. sucking out the liquid from the sampling and/or injecting area 103 or by implementing a sponge or other absorbent materials in the sealing element 110 thereby removing unwanted liquids and preventing the contamination of the sample.

Further, the device 100 comprises a sampling and/or injecting arrangement 105 configured to engage within the sampling and/or injecting area 103, which is sealed off from the rest of the environment by the sealing element 110. FIG. 1 displays a configuration in which the sampling and/or injecting element 107 is disposed within the sampling and/or injecting arrangement 105 together with a piercing element 106, however other configurations may also be possible.

An activator 113 is configured to activate the movement of the sampling and/or injecting arrangement 105 to and from the sampling and/or injecting area 103. The piercing element 106 engages with the surface of the skin 200 at the incision and/or puncture site 108 on the surface of the skin (200) and the sampling and/or injecting arrangement 107 is configured to collect the body fluid (e.g. blood) sample.

In one embodiment, the device 100 is used for sampling a body fluid sample (e.g. blood) and/or injecting a compound (e.g. for medication) into a human or animal body through a body orifice such as a vagina. Suitably, the elongated tubular housing 101 is configured to be a least partly inserted into a body orifice, e.g. by being at least partly bendable in response to maneuvering of the device 100 in tight spaces. Further, the device 100 may comprise a rounded outer surface (as shown in FIG. 1), e.g. with rounded corners or edges, thus allowing for smooth insertion of the device and minimizing the risk of cutting or otherwise damaging e.g. the vagina. More particularly, the device 100 is used for sampling a body fluid (e.g. blood) from a fetus, e.g. during labor, and/or for injecting a compound (e.g. for medication) into a fetus, and the sampling and/or injecting area 103 to which the securing element 102 is configured to attach to is preferably a scalp area or heel area of the fetus. The device 100 may also be used for sampling a body fluid from a neonate and/or injecting a compound into a neonate, with the sampling and/or injecting area 103 being e.g. an area of the scalp or heel of the neonate. Thus, a user, e.g. a midwife, nurse, or doctor, may apply the device 100 to a fetus in utero of a woman as well as apply the device 100 to the surface of the skin 200 of a newborn for sampling a body fluid and/or injecting a compound.

As shown in FIG. 1, the sampling and/or injecting arrangement 105 may comprise a piercing element 106. The piercing element 106 is capable of coming into contact with the sampling and/or injecting area 103 on the surface of the skin 200. The piercing element 106 may be a lancet and/or needle for making an incision in and/or for puncture 108 the sampling and/or injecting area 103. By making an incision and/or puncture 108 with a needle instead of e.g. a cut with a knife as currently employed by the sampling kits available, the site of incision may be reduced, thus allowing for lower invasiveness. Further, as the puncture makes a hole, body fluids such as blood will collect as droplets instead of spreading out, thus allowing for easier and cleaner drawing of a body fluid sample from the incision and/or puncture site 108.

The sampling and/or injecting arrangement 105 may further comprise a sampling and/or injecting element 107, which may be a tube, e.g. a capillary tube or a swab, configured to collect a body fluid sample from the sampling and/or injecting area 103 and/or configured to inject a substance to the sampling and/or injecting area 103.

In the embodiment of FIG. 1, the piercing element 106 and tube 107 extend along a longitudinal axis of the tubular housing 101 and may each be at least partly bendable, e.g. in response to bending of the tubular housing 101 when the device 100 is maneuvered in tight spaces such as a vagina. In FIG. 1, the piercing element 106 and tube 107 extend from the housing 101 into the incision and/or puncture site 108.

The piercing element 106 and/or sampling and/or injecting element 107 may be configured to be partly or fully retractable into and extendable from the tubular housing 101 and/or the sampling and/or injecting arrangement 105, however other configurations are possible. For example, upon activation of the device 100, the piercing element 106 and sampling and/or injecting element 107, are triggered to extend from the tubular housing 101 to the surface of the skin 200, where the piercing element 106 makes an incision and/or puncture at the incision and/or puncture site 108 within the sampling and/or injecting area 103 sealed off by the sealing element 110, for drawing a body fluid sample from the incision site 108 using the sampling and/or injecting element 107. Due to the positioning of the piercing element 106 and the sampling and/or injecting element 107 in relation to each other it can be ensured that they are co-aligned with each other and thus, that the sampling and/or injecting element 107 are automatically steered into the same spot as the incision and/or puncture site 108 for collection of the body fluid sample.

In the embodiment of FIG. 1, a sealing element 110 is disposed in the securing element 102, which sealing element 110 has an aperture for allowing the piercing element 106 and the sampling and/or injecting element 107 to pass through, e.g. when making the incision in the sampling and/or injecting area 103.

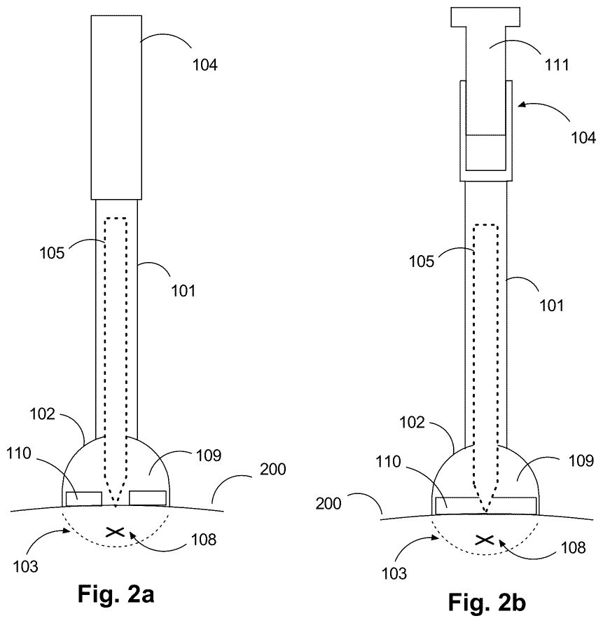

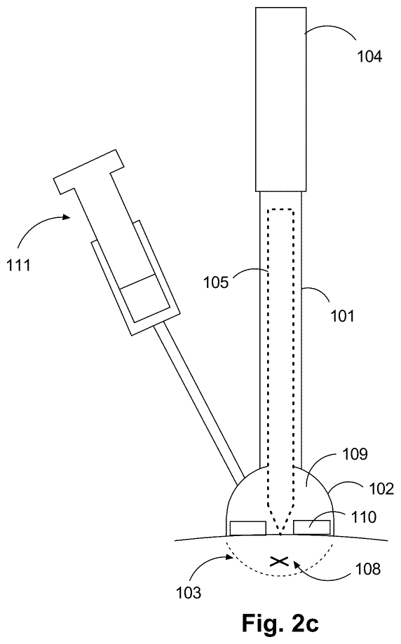

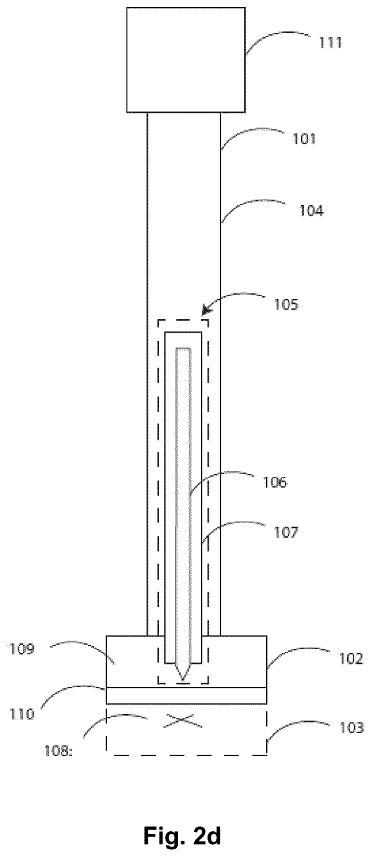

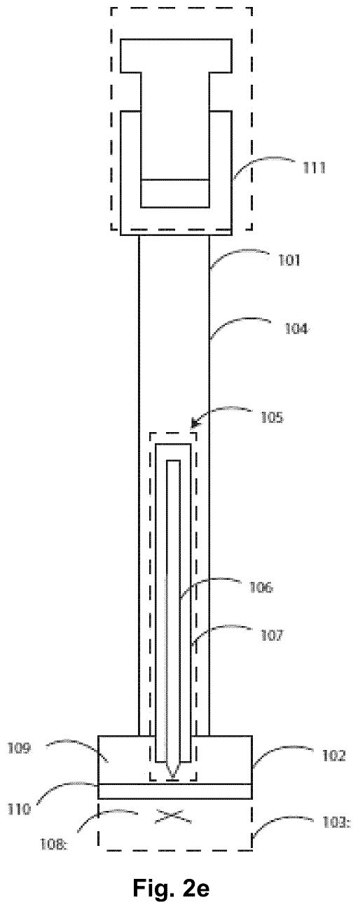

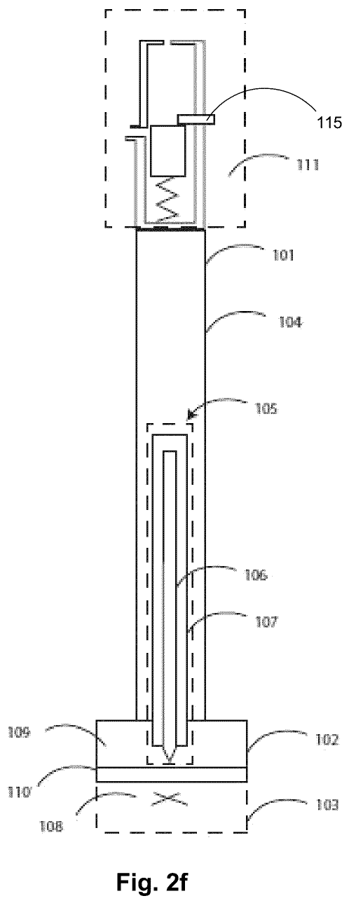

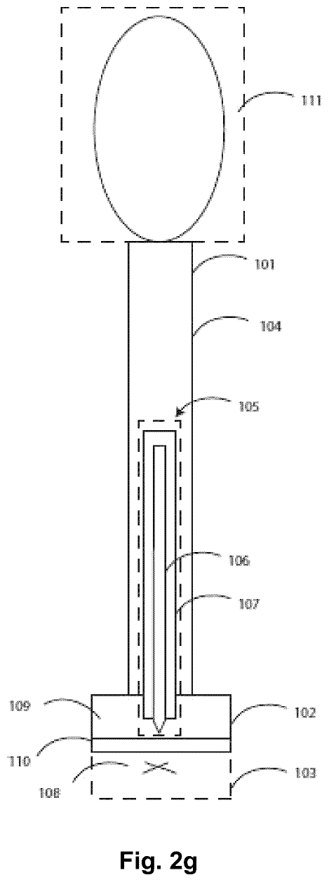

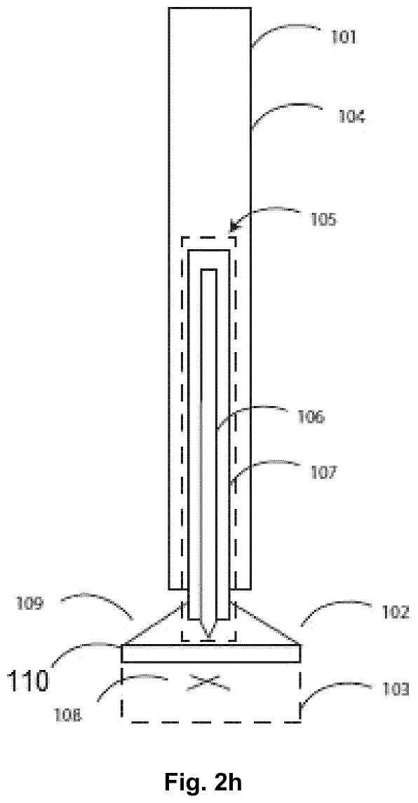

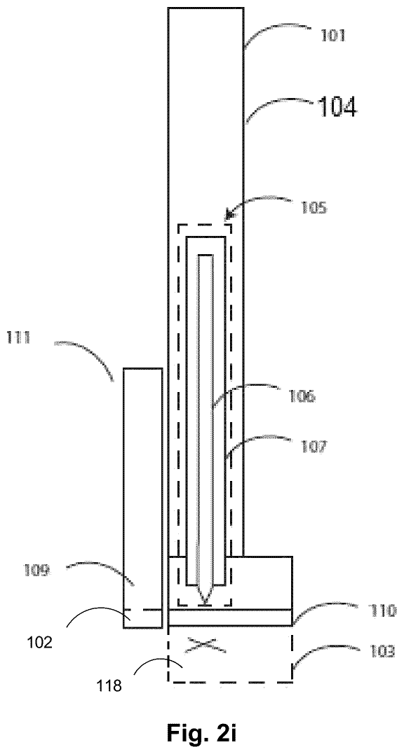

FIGS. 2a to 2i show sampling and/or injecting devices 100 with or without a vacuum generation arrangement 111. FIGS. 2a and 2h show possible configurations for the device 100 without the vacuum generating arrangement 111, using frictional forces to secure the device 100 onto a surface 200. FIGS. 2b, 2c, 2d, 2e, 2f, 2g, and 2i show various possible configurations for the device 100 with a vacuum generating arrangement 111 using vacuum forces to secure the device 100 onto a surface 200.

FIGS. 2a and 2h shows sampling and/or injecting devices 100 without a vacuum generating arrangement 111. The device according to FIG. 2a utilizes frictional forces to adhere to the skin surface 200. A handle 104 may be disposed at the proximal end of the tubular housing 101 and aids the precision insertion of the device into the body orifice e.g. the birth canal. The sampling and/or injecting arrangement 105 is disposed within the tubular housing 101 and comprises the piercing element 106 and the sampling and/or injecting element 107. The securing element 102 may have various forms and shapes, e.g. a cup-shaped outer and/or inner surface, as shown in FIG. 2a, or conical shapes as shown in FIG. 2h. It is understood that any suitable forms or shapes may be used to secure the securing element 102 onto the surface of the skin 200 by means of friction.

FIGS. 2b, 2c, 2d, 2e, 2f, 2g, and 2i show various possible configurations for the vacuum generating arrangement 111. The securing element 102 is operably connected to the vacuum generating arrangement 111 for generating a vacuum between the securing element 102 and the surface of the skin 200. This securely attaches the sampling and/or injecting device 100 to the sampling and/or injecting area 103 by vacuum forces, and, optionally, increases a flow of body fluid to the sampling and/or injecting area 103.

In FIGS. 2b, 2e, and 2g, the vacuum generating arrangement 111 is comprised in the handle 104 of the device. Vacuum is generally generated either pneumatically (ejectors) or electrically (pumps, blowers). A pump module may be used for manually or automatically creating the (partial) vacuum between the securing element 102 and the surface of the skin 200. The vacuum is then applied to all of the sampling and/or injecting area 103 enclosed by the securing element 102 to aid the drawing of the blood droplet.

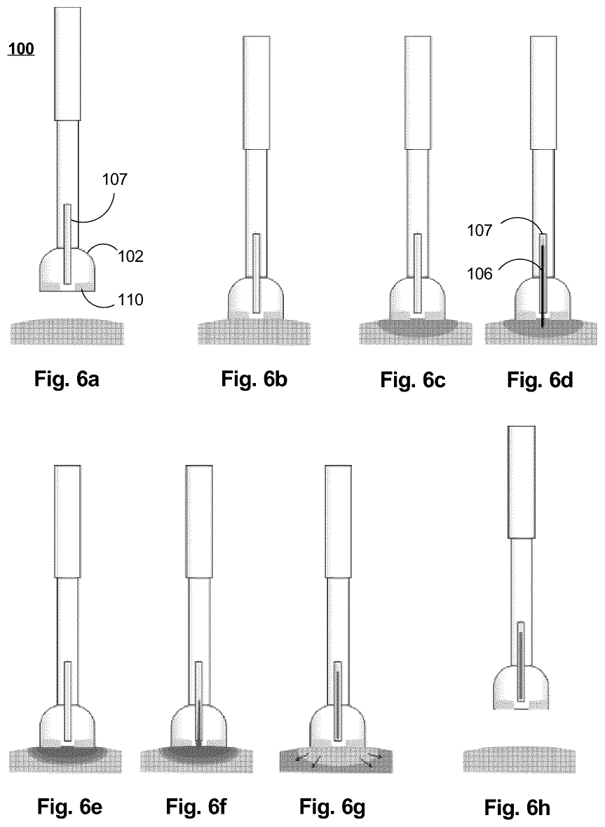

Further, the vacuum may be generated in a first step, wherein the securing element 102 attaches to the sampling and/or injecting area 103, and in a second step, wherein the flow of body fluid to the sampling and/or injecting area 103 is increased (see also FIG. 6c), the second step immediately following said first step.

FIG. 2c shows a configuration where the vacuum generating arrangement 111 is a separate add-on device attached to the device 100 by connection of the vacuum generating arrangement 111 to the securing element 102, e.g. via a valve or vent in the securing element wall. The vacuum generating arrangement 111 may be detachable from the device.

FIG. 2d shows the vacuum generating arrangement 111 that may also be disposed within the tubular housing 101 and the handle 104, hence making this module invisible for the user.

FIG. 2f shows a configuration of the vacuum generating arrangement 111 with the activator 113 and e.g. a mechanical resistor 114 disposed within the tubular housing 101 of the handle 104, operating in a spring-loaded manner to generate vacuum within the cavity 109 created between the surface of the skin 200 and the securing element 102 at the sampling and/or injecting area 103. The sampling and/or injecting arrangement 105 is disposed within the tubular housing 101 and is activated by the vacuum generating arrangement 111 pneumatically or mechanically upon securing the securing element 102 onto the surface of the skin 200 to puncture the surface of the skin 200 at the required incision and/or puncture site to collect a body fluid (e.g. blood) for analysis. Alternatively, the activator 113 first activates the vacuum generating arrangement 111 and a subsequent engagement of the activator 113 then activates the sampling and/or injecting arrangement 105 to engage with the sampling and/or injecting area 103.

FIG. 2i shows a configuration of the vacuum generation arrangement 111 in which is it disposed near the outside of the tubular housing 101 with the sealing element 110 disposed at the end of the vacuum generating arrangement 111. The vacuum generating arrangement 111 can be either permanently attached to the surface of the tubular housing 101 or may be removed, depending on the configuration. The securing element 102 may also be placed near the sampling and/or injecting area 103. The sampling and/or injecting arrangement 105 may further comprise of a sealing element 110, configured to seal the sampling and/or injecting area 103 to avoid contamination of the sample.

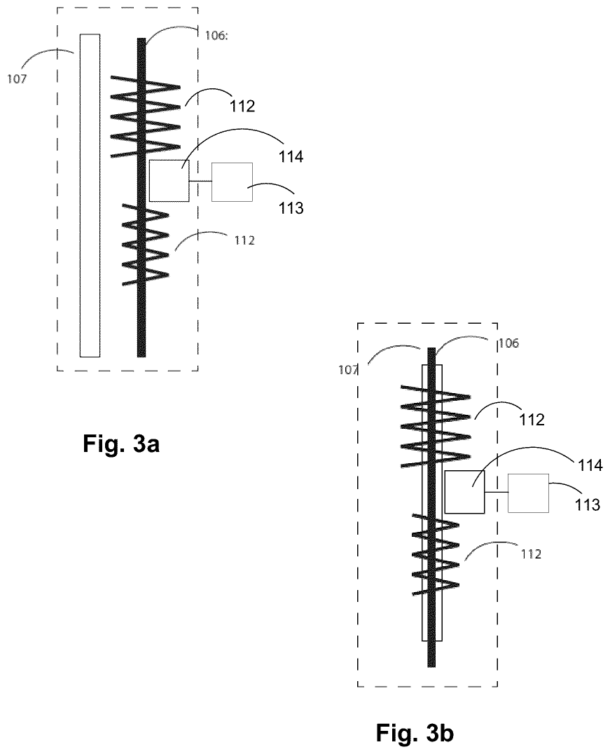

FIGS. 3a and 3b show the possible disposition of the sampling and/or injecting element 107 disposed near the springs 112 for controlled activation and de-activation of the device to and from the sampling and/or injecting area 103. The activator 113 is mechanically operably connected to the mechanical resistor 114.

FIG. 3a shows a possible configuration where the sampling and/or injecting element 107 is adjacent to the mechanical resistor 114 and coupled to the activator 113 and springs 112 within the sampling and/or injecting arrangement 105. The activator 113 and mechanical resistor 114 is mechanically operably connected adjacent to the sampling and/or injecting element 107. It is also possible to dispose the sampling and/or injecting element 107 around the piercing element 106, i.e. so that the piercing element 106 is disposed within the sampling and/or injecting element 107, whereby the mechanical resistor 114 is mechanically connected to the activator 113.

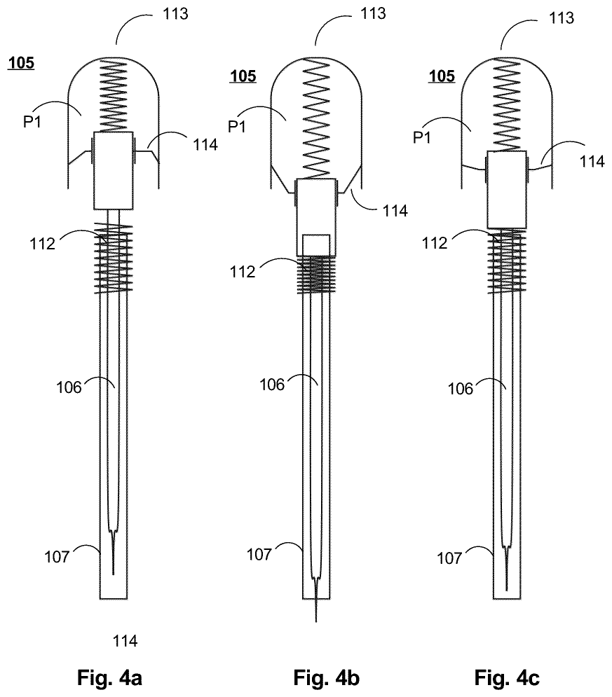

FIGS. 4a to 4c show another possible configuration of the sampling and/or injecting arrangement 105 of a sampling and/or injecting device 100. The piercing element 106 and sampling and/or injecting element, preferably tube, 107 are operably coupled to one-another. The piercing element 106 and tube 107 are supported in the tubular housing (not shown) in a spring-loaded manner by a first spring element 112 and a second spring element 113. The mechanical resistor 114 is pneumatically connected to the activator 113. FIG. 4a shows the sampling and/or injecting arrangement in an inactive mode, with the piercing element 106 retracted into the tube 107. Upon activation of the piercing element 106 for making an incision and/or for puncturing a sampling and/or injecting area, the piercing element 106 is automatically triggered to extend from the tube 107, as shown in FIG. 4b. Activation may occur e.g. when the user of the sampling and/or injecting device presses the handle 104 relative to the tubular housing 101 as e.g. a pump, or presses a button (e.g. an activator 113) at a proximal end of the tubular housing 101. A mechanical resistor 114 is placed in the tubular housing and is operatively connected to the activator 113, creating a cavity P1 within the sampling and/or injecting arrangement 105. By activating the vacuum generator 111, internal pressure within the cavity P1 portion of the sampling and/or injecting arrangement (105) changes and created a vacuum between the surface of the skin 200 and the sampling and/or injecting arrangement 105. The mechanical resistor 114 may be a membrane, configured to pneumatically move to a pre-configured extent so that the piercing element 106 can make an incision. When pressure is lowered in the cavity P1 of the tubular housing 101 and hence the mechanical resistor 114 collapse due to pressure difference.

After incision and/or puncturing, allowing the tube 107 to automatically collect a body fluid sample from the incision and/or puncture site using the capillary effect, the piercing element 106 may automatically retract back into the tube 107 (as shown in FIG. 4c.)

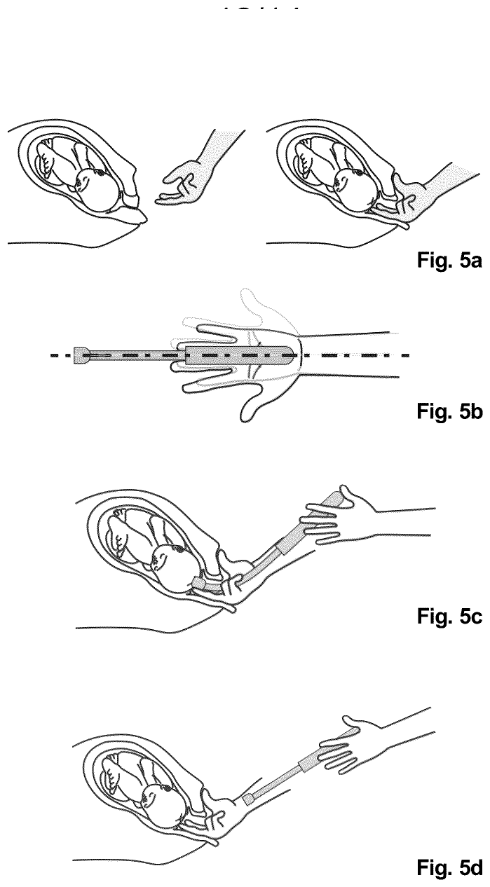

FIGS. 5a to 5d are diagrammatic illustrations of a method for fetal scalp blood sampling during labor, wherein the sampling and/or injecting device disclosed herein is used. In FIG. 5a, the position of the fetus is assessed by a user of the device, typically a midwife or doctor. In the present example, the assessment involves insertion of two fingers (left or right hand of choice) into the birth canal for ensuring that the fetus is positioned with its scalp at the cervix.

FIG. 5b shows the user picking up the device with his/her free hand. In an embodiment, the device 100 is symmetrical along its longitudinal axis. In that case there is no wrong way for the user to pick up the proximal end of the device/the handle.

FIG. 5c shows the user steering the securing element disposed at the distal end of the tubular housing 101 of the device, and at least part of the tubular housing, into the vagina for mounting the securing element 102 on the scalp of the fetus.

The area of the fetal scalp enclosed by the sealing element 110 defines the area of sampling. For steering, the user may use his/her arm as guidance, as shown. A small fin (not shown), functioning as a finger grip, may be provided close to or on the securing element 102 for helping the user to get a grip of the securing element 102 once inside of the vagina, for better control.

In an embodiment, the mounted securing element 102 attaches securely to the fetal scalp by friction forces between the securing element and the sampling and/or injecting area 103. In another embodiment, the securing element 102, additionally or instead, attaches by vacuum forces resulting from a (partial) vacuum created in the securing element, the securing element 102 thereby functioning as a suction cup. When the securing element 102 is properly attached, the user may start the puncture-and-collecting process, as described in connection with FIGS. 6a to 6h. Once a fetal blood sample has been drawn, the securing element 102 releases from the fetal scalp, and the user extracts/pulls out the device from the vagina, as shown in FIG. 5d. Following extraction, the device or at least a part of it, e.g. the piercing element 106 and/or sampling and/or injecting element (107), may be disposed.

FIGS. 6a to 6h are diagrammatic illustrations of a method for collecting a body fluid sample using the sampling and/or injecting device 100 disclosed herein. Particularly, the steps of mounting the device on an inner or outer body surface (FIGS. 6a, 6b), securely attaching the securing element 102 to a sampling and/or injecting area (FIG. 6c) of the body surface using friction and/or vacuum, making an incision in and/or puncturing the sampling area (FIG. 6d), collecting a body fluid sample into the tube 107 (FIGS. 6e, 6f), and releasing the securing element 102 from the sampling and/or injecting area (FIGS. 6g and 6h) are shown.

During mounting, the securing element 102 may be moved over an area including the sampling and/or injecting area, thereby letting the sealing element 110 disposed at the distal end of the device 100 to e.g. absorb liquid fluids on the body surface in order to clean the area e.g. sucking out the liquid from the sampling and/or injecting area 103 or by implementing a sponge or other absorbent materials in the sealing element 110 thereby removing unwanted liquids and preventing the contamination of the sample.

Thus, once the securing element 102 is mounted on, and securely attached to, the body surface, the sampling and/or injecting area enclosed by the securing element 102 will be clean, and liquid inflow from the surroundings can be prevented. Puncturing and sampling may thus be performed in a clean environment. The sponge 110 may as well serve to minimize or prevent liquid inflow into the securing element 102 and, thus, also into the lumen of the tubular housing 101 during entry (or exit) of the device into (or from) a body orifice such as a vagina. Further, moving of the sponge 100, during mounting, over an area containing the sampling and/or injecting area may stimulate hyperemia, thus ensuring that a sufficient volume of blood can be drawn.

Instead of the sponge 110, a thin membrane or film may be provided at the open end of the securing element 102, sealing off the inner of the securing element 102. During the puncture and collect phase, the piercing element 106 pierces through the membrane or film before making an incision in the sampling and/or injecting area.

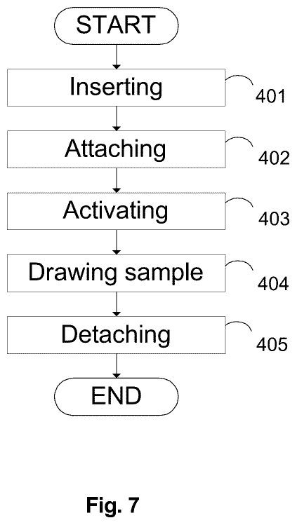

FIG. 7 is a flowchart of a method for collecting a fetal and/or neonatal body fluid sample during labor, using the sampling and/or injecting device disclosed herein. In step 401 (optional), the device is inserted into a body orifice of a patient. In step 402, the securing element 102 is securely attached to the sampling area on the fetus or neonate. In step 403, an incision and/or puncture is made in tissue of the sampling area. In step 404, a body fluid sample is drawn from the incision or puncture site into the tube of the sampling and/or injecting device for collecting a fetal and/or neonatal body fluid sample, which sample may be used for determining and analyzing Lactate and/or oxygen levels therein in order to test whether oxygenation is sufficient. In step 405, the securing element is released from the sampling area.

The various aspects and implementations have been described in conjunction with various embodiments herein. However, other variations to the disclosed embodiments can be understood and effected by those skilled in the art in practicing the claimed subject-matter, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measured cannot be used to advantage.

The reference signs used in the claims shall not be construed as limiting the scope. Unless otherwise indicated, the drawings are intended to be read together with the specification and are to be considered a portion of the entire written description of this disclosure.

1. A sampling and/or injecting device comprising
an elongated tubular housing, which tubular housing is at least partly bendable, and
a sampling and/or injecting arrangement configured to engage a sampling and/or injecting area, said sampling and/or injecting arrangement comprising
a piercing element configured to engage said sampling and/or injecting area, and
a sampling and/or injecting element configured for automatically collecting a body fluid sample from said sampling and/or injecting area and/or configured for injecting a substance in said sampling and/or injecting area,
characterised in that said sampling and/or injecting device further comprises
a securing element disposed at a distal end of said tubular housing, which securing element is operably connected to a lumen of said tubular housing and configured to attach with an open end to a sampling and/or injecting area,
a sealing element disposed within or around said securing element and configured to seal said sampling area,
an activator configured to activate automatic movement of said piercing element to and from said sampling and/or injecting area, after attachment of said securing element to said sampling and/or injecting area, and
a mechanical resistor for controlling said automatic movement of said piercing element, said mechanical resistor being mechanically or pneumatically operably connected to said activator.

2. The device according to claim 1, wherein said securing element and said elongated tubular housing are configured to be at least partly inserted into a body orifice.

3. The device according to claim 1, wherein said device is configured for sampling a body fluid from a fetus and/or neonate, and/or for injecting a compound into a fetus and/or neonate, and wherein said sampling and/or injecting area is one of a scalp area and a heel area of said fetus or neonate.

4. The device according to claim 1, wherein said piercing element is a lancet or a needle for making an incision in and/or for puncturing said sampling and/or injecting area.

5. The device according to claim 1, wherein said sampling and/or injecting element is a tube, preferably a capillary tube, or a swab configured for collecting a body fluid sample from said sampling and/or injecting area, and/or a tube configured for injecting a substance in said sampling and/or injecting area.

6. The device according to claim 1, wherein said sampling and/or injecting element is provided at least partly within said tubular housing.

7. The device according to claim 1, wherein said sampling and/or injecting element is configured for guiding said piercing element.

8. The device according to claim 1, wherein said sealing element has an aperture for allowing said piercing element and/or said sampling and/or injecting element to pass through to the sampling and/or injecting area.

9. The device according to claim 1, wherein said securing element is configured to attach to said sampling and/or injecting area by means of friction forces between said securing element and said sampling and/or injecting area.

10. The device according to claim 1, further comprising a vacuum generating arrangement for generating a vacuum within said securing element, said vacuum generating arrangement optionally being activated by said activator.

11. The device according to claim 10, wherein said vacuum generating arrangement is configured to attach said securing element to said sampling and/or injecting area, and, optionally, to increase a flow of body fluid to said sampling and/or injecting area by vacuum forces.

12. The device according to claim 11, wherein said vacuum generating arrangement is configured such that said vacuum is generated in a first step, attaching said securing element to said sampling and/or injecting area, and in a second step, after said sampling and/or injecting arrangement engaging the sampling and/or injecting area, increasing said flow of body fluid to said sampling and/or injecting area by means of said vacuum, said second step immediately following said first step.

13. The device according to claim 10, wherein said vacuum is applied to all of said sampling and/or injecting area enclosed by said securing element.
